SEQUENCE LISTING (2 sequences)

SEQ ID NO: 1 - DNA, 17 bp, Artificial Sequence
  Synthetic Oligonucleotide
ggacggcggctttgttg

SEQ ID NO: 2 - DNA, 21 bp, Artificial Sequence
  Synthetic Oligonucleotide
cctcggtggcacggcggatgt

(19) United States
(12) Patent Application Publication
(10) Pub. No.: US 2004/0265340 A1
(43) Pub. Date: Dec. 30, 2004

(54) Emulsan adjuvant immunization formulations and use

(75) Inventors: David L. Kaplan, Stow, MA (US); Juliet Fuhrman, Arlington, MA (US); Richard A. Gross, Plainview, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignees: Trustees of Tufts University, Medford, MA; University of Massachusetts, Lowell, MA

(21) Appl. No.: 10/767,836

(22) Filed: Jan. 29, 2004

Related U.S. Application Data
(63) Continuation of application No. 09/518,020, filed on Mar. 3, 2000, now abandoned.
(60) Provisional application No. 60/123,056, filed on Mar. 5, 1999.

(51) Int. Cl. A61K 39/02

(52) U.S. Cl. 424/234.1; 424/200.1

(57) ABSTRACT

Immunization formulations comprising an antigen and an emulsan or emulsan analog are formed and can be administered to a host. The emulsan or emulsan analog is an adjuvant in the immunization formulation. The emulsan or emulsan analog is secreted from Acinetobacter calcoaceticus. In particular, the emulsan or emulsan analog is secreted from Acinetobacter calcoaceticus RAG-1. The emulsan analog is produced and secreted from Acinetobacter calcoaceticus cultured in the presence of varying fatty acid sources. The emulsan analog is also produced and secreted from mutants of Acinetobacter calcoaceticus, such as transposon mutants of Acinetobacter calcoaceticus RAG-1.

### RELATED APPLICATION

This application is a continuation of U.S. application Ser. No. 09/518,020, filed on Mar. 3, 2000, which claims the benefit of U.S. Provisional Application No. 60/123,056, filed on Mar. 5, 1999. The entire teachings the above applications are incorporated herein by reference in their entirety.

### GOVERNMENT SUPPORT

The invention was supported, in whole or in part, by a grant 96355003190 from Department of Agriculture. The Government has certain rights in the invention.

### BACKGROUND OF THE INVENTION

Cytokine activity has been a subject of intense research investigation. For example, adjuvants have been combined with antigens and injected into a mammal (e.g., human, mouse, rabbit, sheep) to generate an immune response (e.g., antibody production, Tumor Necrosis Factor (TNF) release from macrophages) which, in the case of vaccines, provides protective immunity to the mammal. A suitable adjuvant has the capacity to generate an immune response with minimal side effects (e.g., fever, arthritis, granulomas) to the mammal. In humans, adjuvants are generally limited to aluminum based adjuvants which generate immune responses yet are a serious health concern due to the suggested role of aluminum in Alzheimer's disease (Allison, et al., J. Immunol. Methods 95:157-68(1986), the teachings of which are incorporated herein by reference in their entirety).

Many adjuvants suffer from a number of limitations. For example, limitations include a failure of the adjuvant to induce an appropriate antibody and T-cell response, including a failure to result in the release of cytokine (e.g., TNF); and inadequate shelf life leading to unstable and degraded forms of the adjuvant.

Thus, a need exists to develop new adjuvants that minimize or overcome the above stated problems.

### SUMMARY OF THE INVENTION

The present invention relates to emulsan or emulsan analogs for use as an adjuvant in immunization formulations.

In one embodiment, the immunization formulation comprises an antigen and an emulsan or an emulsan analog. In a preferred embodiment, the emulsan or emulsan analog is secreted from Acinetobacter calcoaceticus. A more preferred embodiment, the emulsan or emulsan analog is secreted from Acinetobacter calcoaceticus RAG-1.

In yet another embodiment, the emulsan analog is secreted by a mutant of Acinetobacter calcoaceticus. In a particular embodiment, the emulsan analog is secreted by a transposon mutant of Acinetobaceter calcoaceticus.

In a preferred embodiment, the emulsan analog has a fatty acid chain length in a range of about 10 carbons and about 20 carbons. In one embodiment, the emulsan analog has a fatty acid density in a range of about 25 nmol/mg emulsan and about 900 nmol/mg emulsan. In another embodiment, the emulsan analog has an amount of saturated fatty acid bonds in fatty acids of the analog in a range of about 80 mole % and about 100 mole %. In yet another embodiment, the emulsan analog has an amount of hydroxylated fatty acid in a range of between about 0 mole % and about 65 mole %.

In a particular embodiment, the emulsan analog is formed by feeding Acinetobaceter calcoaceticus or a mutant thereof a compound selected from the group consisting of fatty acids, fatty acid salts, hydroxylated fatty acid salts and complex carbon sources that include fatty acids, said group having a carbon chain length in a range of between about 10 carbons and about 20 carbons.

In a preferred embodiment of the invention, the antigen used in the immunization formulation is selected from a group consisting of peptides, polypeptides, viruses, bacteria, fungi, parasites, and any combination or fragment thereof.

Another aspect of the invention relates to a method of stimulating cytokine release in a host, comprising the step of administering to the host an emulsan or an emulsan analog. In a preferred embodiment, the cytokine release is accompanied by immunomodulation of the host. In one embodiment, the host is a cell line. In another embodiment, the host is a mammal.

The invention further relates to a method of producing an emulsan analog, comprising the steps of mutating Acinetobaceter calcoaceticus by transposon mutagenesis to form Acinetobaceter calcoaceticus mutants and feeding at least one of the mutants a compound selected from the group consisting of fatty acids, fatty acid salts, hydroxylated fatty acid salts and complex carbon sources that include fatty acids, said group having a carbon chain length in a range of between about 10 carbons and about 20 carbons.

In another embodiment, the invention relates to a formulation comprising an antigen and an emulsan for stimulating an immune response in an organism. In yet another embodiment, the invention relates to a formulation comprising an antigen and an emulsan analog for stimulating an immune response in an organism. Examples of suitable organisms include mammals, birds and ruminants.

The invention described herein provides an immunization formulation for administering an antigen and generating an immune response in a host. Advantages of the claimed invention include, for example, immunization formulations comprising emulsan or emulsan analogs as adjuvants which elicit immune response. The formulation can have significantly fewer adverse side reactions and be produced more cost effectively than other adjuvant formulations. The claimed immunization formulations of the invention provide readily available sources to administer antigens for antibody production and for use in vaccines. The emulsan or emulsan analog adjuvants described herein can be synthesized directly from microbial sources with a high degree of control over structural features, and be purified by relatively simple procedures. Thus, adjuvants which include emulsan or emulsan analogs can combine a desired immune response, low organismal and cellular toxicity, as well as efficient delivery and release properties.

### BRIEF DESCRIPTION OF THE FIGURES

FIG. 1 depicts an illustration of an emulsan repeating motif.

FIG. 2 depicts the strategy for generating transposon mutants.

FIG. 3A depicts the fatty acid profile (mole %/mg emulsan of transposon mutant 2 cultured in the presence of Luria Broth alone and Luria Broth with undecanoic acid (C11 1% w/v) as a carbon source.

FIG. 3B depicts the fatty acid profile (mole %/mg emulsan of transposon mutant 2 cultured in the presence of Luria Broth alone and Luria Broth with myristic acid (C14 1% w/v) as a carbon source.

FIG. 4A depicts the emulsification activity of transposon mutant 1 grown in Luria Broth (LB) alone and Luria Broth with fatty acid of varying carbon lengths (C11, C14, C16, C18).

FIG. 4B depicts the emulsification activity of transposon mutant 2 grown in the presence of Luria Broth (LB) alone and Luria Broth with fatty acid of varying carbon lengths (C11, C14, C16, C18).

FIG. 5A depicts TNF release by murine primary macrophages, also referred to herein as peritoneal macrophages stimulated by lipopolysaccaride (LPS), crude emulsan (EM) or deproteinized emulsan.

FIG. 5B depicts TNF release by RAW 264.7 cells stimulated with emulsan (EM).

FIG. 6 depicts the nitrite release from emulsan (EM) stimulated RAW 264.7 cells compared to lipopolysaccaride (LPS) and media alone.

FIG. 7A depicts TNF release in response to crude sophorolipid (SL), emulsan (EM), ethyl ester of sophorolipid (C2), and diacetylated ethyl ester of the sophorolipid (Ac) in HeNC2 cells or GG2EE cells.

FIG. 7B depicts TNF release in response to lipopolysaccharide (LPS) in HeNC2 cells and GG2EE cells.

FIG. 8 depicts the fatty acid content of emulsan (EM1) and various emulsan analogs EM4-EM10).

FIG. 9A depicts TNF release from macrophages stimulated by emulsan and emulsan analogs (EM1-EM10) with varying degrees of fatty acid substitution (nmol of fatty acids per mg of emulsan).

FIG. 9B depicts the relationship between fatty acid content of emulsan and emulsan analogs on TNF release by macrophage cells.

FIG. 10 depicts the kinetics of emulsan activity after in vivo administration.

FIG. 11A depicts the levels of IgG1 in response to antigen (100 μg DNP-KLH) (Ag), emulsan alone (200 μg) (EM), deproteinized emulsan alone (200 μg) (Dp-EM), emulsan at a relatively low concentration (20 μg) (EM low and Ag), emulsan at a relatively high concentration (200 μg) (EM high and Ag), deproteinized emulsan at a relatively low concentration (20 μg) (Dp-EM low and Ag), deproteinized emulsan at a relatively high concentration (200 μg) (Dp-EM and Ag) or complete Freund's adjuvant mixed with incomplete Freund's adjuvant and antigen 100 μg DNP-KLH (CFA/IFA & antigen) nine days after immunization.

FIG. 11B depicts the levels of IgG2a in response to antigen (100 μg DNP-KLH) (Ag), emulsan alone (200 μg) (EM), deproteinized emulsan alone (200 μg) (Dp-EM), emulsan at a relatively low concentration (20 μg) (EM low and Ag), emulsan at a relatively high concentration (200 μg) (EM high and Ag), deproteinized emulsan at a relatively low concentration (20 μg) (Dp-EM low and Ag), deproteinized emulsan at a relatively high concentration (200 μg) (Dp-EM and Ag) or complete Freund's adjuvant mixed with incomplete Freund's adjuvant and antigen 100 μg DNP-KLH (CFA/IFA & antigen) nine days after immunization.

FIG. 12 depicts an antibody titration curve of antigen alone (Group 1) and various preparations of emulsan (Groups 2-7) and adjuvant (Group 8) 9 days following immunization. Groups 1-8 are defined in Table 6.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention relates to emulsan or emulsan analogs used as adjuvants in immunization formulations. The emulsan or emulsan analogs stimulate cytokine release in a host without inducing toxicity to the host. The emulsan or emulsan analog can be combined with an antigen to generate an immune response in the host which can, for example, result in the production of antibodies to the antigen or provide prophylaxis against the antigen. Thus, the emulsan or emulsan analogs of the invention are effective adjuvants.

An “immunization formulation” refers to any preparation of an antigen, an emulsan, emulsan analog, or any combination thereof, capable of generating an immune response. An immune response can be, for example, the production of antibodies to the antigen, the release of cytokines (e.g., TNF) from cells (e.g., macrophages), or both. Techniques to evaluate immune responses are well known to one of skill in the art and include, for example, ELISA, RIA, Ouchterlony plates and immunodiffusion analysis. (See, for example, Ausubel et al., “Current Protocols in Molecular Biology” John Wiley & Sons, (1998); Coligan, et al., “Current Protocols in Immunology” John Wiley & Sons (1991), the teachings of which are incorporated herein by reference in their entirety).

The emulsan or emulsan analog, by facilitating or enhancing an immune response to an antigen, is an adjuvant. An “adjuvant” refers to a composition (e.g., emulsan or emulsan analog) which elicits immune responses to antigens. An adjuvant can, for example, when mixed with an antigen, form a depot in tissues from which the antigen can be released. Additionally, or alternatively, an adjuvant can stimulate immune mediating cells (e.g., B lymphocytes, T lymphocytes or both) to enhance an immune response. For example, an adjuvant can attract or stimulate T- and/or B-lymphocytes to an area of antigen deposition thereby eliciting or stimulating an immune response (e.g., antibody production) to the antigen. The emulsan or emulsan analogs described herein are used as adjuvants.

The emulsan or emulsan analogs may be employed to stimulate immune responses (e.g., the production, release and/or activity of cytokines, such as TNF from macrophages), in the absence of antigens. When employed to stimulate immune responses in the absence of an antigen, the emulsan or emulsan analog is also referred to herein as a “stimulant.”

The immunization formulation can be a mixture of the antigen, emulsan or emulsan analog, or any combination thereof, in any concentration or ratio. For example, the antigen, emulsan and emulsan analog can be combined at a concentration ratio of 1:1:1, 1:2:1, 5:1:1, or 2:1:1. Likewise, when emulsan alone or emulsan analog alone is used in combination with an antigen in the immunization formulation, the emulsan:antigen or emulsan analog:antigen concentration ratio can be, for example, 1:5 or 2:1.

Antigens generally induce a state of sensitivity, an immune response, or both. The state of sensitivity and immune response can be evaluated in a host (e.g., a cell, collection of cells such as a macrophage cell line or an animal cytokine production, release, activity) using standard techniques. (Coligan, et al., “Current Protocols in Immunology” John Wiley & Sons (1991)). The antigens can result in the production, also referred to herein as generation, of antibodies when injected (e.g., intramuscularly, subcutaneously or intraperitoneally) or ingested (e.g., orally) into a host. The terms hapten and immunogen can be used interchangeably with the term antigen.

Suitable antigens can be peptides, polypeptides, glycoproteins, bacteria, virus, fungi, parasites, small organic molecules, lipids, simple or complex carbohydrates, nucleic acids, or any combination thereof. The antigen can be a naturally occurring antigen (e.g., obtained or isolated from nature). Additionally, or alternatively, the antigen can be normaturally occurring (e.g., a synthetic peptide or recombinantly produced polypeptide).

It is further envisioned that fragments or portions of antigens can be used in the immunization formulations of the invention. A “fragment” or a “portion” of antigen is any part of the antigen which is less than the total, complete or full antigen. For example, a fragment of a polypeptide antigen can be the amino acids comprising the carboxy terminus of the polypeptide, the amino acids comprising the amino terminus of the polypeptide or amino acids of a particular portion of the polypeptide. Similarly, a fragment or a portion of a bacteria or viral antigen can be the plasma membrane or cellular fraction of the virus or bacteria.

When cells such as bacteria, fungi, viruses, or parasites are used as antigens, the cells can be alive or dead. Dead cells for use as an antigen can be produced by a variety of suitable methods well known to one of skill in the art, including heat or chemically induced or facilitated death. The cells can also include attenuated, also referred to as variant or mutant, forms of the bacteria, virus, fungi or parasite. An attentuated bacteria, virus, fungi or parasite can have diminished virulence (e.g., infectivity) compared to the unattentuated bacteria, virus, fungi or parasite. Techniques to produce and characterize attentuated bacteria, viruses, fungi or parasites are established and well known to one of skill in the art.

The antigen can optionally be associated, also referred to herein as linked, to an additional carrier molecule such as bovine serum albumin (BSA) or keyhole limpet hemocyanin (KLH). It is also envisioned that other molecules, synthetic or naturally occurring can be associated chemically or mixed with the antigen.

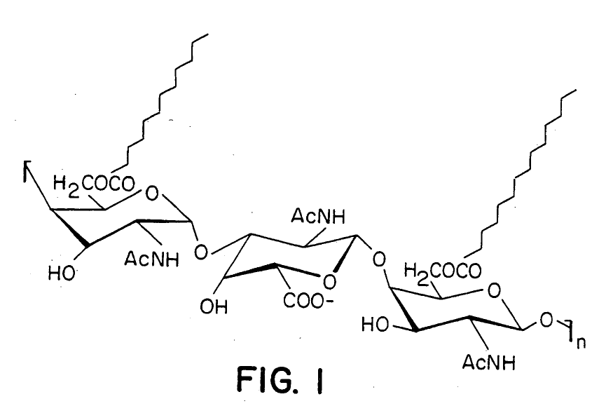

The representative structure of the repeating motif of an emulsan or emulsan analog for use in the invention is depicted in FIG. 1. Emulsan and emulsan analogs are characterized as polyanionic amphiphathic lipoheteropolysaccharides whose main chain or backbone has been reported to include three amino sugars: D-galactoseamine, D-galactosaminuronic acid and diamino-6-deoxy-D-glucose. Fatty acids are covalently linked by N-acyl and O-ester bonds to the backbone of the emulsan or emulsan analog.

Emulsan and emulsan analogs of the invention are extracellular products (e.g., secreted) of a bacterium, such as the gram negative bacterium Acinetobacter calcoaceticus (A. calcoaceticus). In a particular, the emulsan or emulsan analogs are secreted from A. calcoaceticus. Secreted refers to release from an intracellular compartment (e.g., cytoplasm, secretory granules) to the outside of the cell, for example, into the culture media. “Emulsan” as defined herein, are groups of polyanionic amphiphathic lipoheteropolyaccharides secreted by Acinetobacter calcoaceticus RAG-1 when fed ethanol. (FIG. 1).

As used herein, the term “emulsan analog” refers to structural analogs of the group of emulsans as defined above. Emulsan and emulsan analogs are produced by growing A. calcoaceticus in the presence of a carbon source as described above. For example, the carbon source can vary based on the type and concentration of fatty acid side chains, the total fatty acid content, the average density of fatty acids along the polysaccharide backbone, the degree of saturation present in the fatty acid side chains, the degree of substitution (e.g., the presence or absence of hydroxylated fatty acid chains) and by other parameters known in the art.

As used herein, the term “emulsan analog” also refers to emulsans obtained from A. calcoaceticus mutants (e.g., transposon mutants) which can be, for example, emulsans obtained by mutants grown on ethanol as well as mutants grown on carbon sources other than ethanol (e.g., ethyl propionate). Emulsan and emulsan analogs obtained from Acinetobacter S. ATCC 31012 or from mutants thereof are described for example in U.S. Pat. No. 4,311,829, the teachings of which are incorporated herein by reference in their entirety.

In a preferred embodiment of the invention, emulsan and emulsan analogs are obtained from A. calcoaceticus strain RAG-1 or from transposon mutants of A. calcoaceticus RAG-1. Emulsan and emulsan analogs can be obtained from Acinetobacter Sp. ATTC (American Type Culture Collection) 31012 and mutants thereof as described in U.S. Pat. No. 4,311,829 by Gutnick et al. (1982), the teachings of which are incorporated herein by reference in their entirety. Additionally, or alternatively, emulsan analog also includes emulsans having structures such as shown in FIG. 1 which might be obtained from bacteria other than A. calcoaceticus. Emulsan or emulsan analogs also can be synthesized chemically in the absence of a bacterial cell and used in the immunization formulations described herein.

Carbon sources which can be employed in culture medium of emulsan- or emulsan analog-producing bacteria include but are not limited to ethanol, ethyl propionate, saturated or unsaturated fatty acids, salts thereof, hydroxylated fatty acids and complex carbon sources such as, for example, petroleum and petroleum fractions. In a preferred embodiment, the carbon source includes fatty acids having from about 10 carbon atoms (C10) to about 20 carbon atoms (C20), salts thereof, alkyl esters and in particular methyl esters thereof, hydroxylated C10 to C20 fatty acids, any combinations thereof as well as complex carbon sources including C10 to C20 fatty acids or derivatives or combinations thereof. Both saturated and unsaturated C10 to C20 fatty acids can be employed. Preferred examples of fatty acids include decanoic, lauric, tridecanoic, myristic, pentadecanoic, palmitic, heptadecanoic and stearic acids. These are abbreviated herein as 11:0 (11 carbon atoms), 12:0 (12 carbon atoms), 13:0 (13 carbon atoms), 14:0 (14 carbon atoms), 15:0 (15 carbon atoms), 16:0 (16 carbon atoms), 17:0 (17 carbon atoms) and 18:0 (18 carbon atoms), respectively. In yet another embodiment of the invention, the carbon source includes alkyl esters and in particular methyl esters derived from the above 11:0, 12:0, 13:0, 14:0, 15:0, 16:0, 17:0 and 18:0 fatty acids. In a further embodiment, the carbon source includes hydroxylated, in particular 2-hydroxyl 11:0, 12:0, 13:0, 14:0, 15:0, 16:0, 17:0 and 18:0 fatty acids.

As used herein, “complex carbon source” refers to mixtures which include but are not limited to hydrocarbons, carboxcylic acids, derivatives and salts of carboxcylic acids, alcohol, such as, for example carbon sources generally used in growing emulsan and emulsan analogs. Specifically, “complex carbon source” refers to crude oil, petroleum fractions or agricultural oils (e.g., safflower oil).

Measuring units employed herein to describe emulsan and emulsan analogs include mole % (also referred to herein as mol % or mole %), as nanomoles per milligram of emulsan or as % w/w. For example, the 10:0 and 16:0 fatty acid content of emulsan produced by growing A. calcoaceticus RAG-1 in an ethanol carbon source are 2.6 mol % (or 11 nmol/mg) and 23 mol % (or 99 nmol/mg), respectively. Additional values for other side chain fatty acids of emulsan obtained by growing A. calcoaceticus RAG-1 in ethanol are given in Table 1.

In one embodiment of the invention, the emulsan analog has a total fatty acid content in the range of between about 25 nmol/mg and about 900 nmol/mg emulsan. In another embodiment of the invention, the emulsan analog has a fatty acid content in the range of between about 25 nmol/mg emulsan and about 9000 nmol/mg emulsan. “Fatty acid content” (nmol/mg) refers to the nanomoles (nmol) of fatty acid per milligram (mg) of emulsan. The term “Fatty acid content” is used herein interchangeably with the term “fatty acid density.”

In a further embodiment, the emulsan analog has an average content of saturated bonds in fatty acids side chains of the analog in the range of between about 80 mol % and about 100 mol %. In yet another embodiment, the emulsan analog has an average content or amount of hydroxylated fatty acids in a range of between about 0 mol % and 65 mol %.

Emulsan and emulsan analogs having specific structural features can be prepared and characterized as further described below. For example, manipulation in both the composition and degree of fatty acid substitution can be achieved as described in Gorkovenko et al., Proc. Am. Chem. Soc., Div. Polym. Sci. Eng 72:92-93 (1995), Gorkovenko et al., Can. J Microbiol. 43:384-390 (1997), (Gorkovenko, et al., Carbohydrate Polymers, 39:79-84 (1999) and Zhang, et al., J. Biol. Macromol. 20: 9-21 (1997), the teachings of which are incorporated herein by reference in their entirety. Emulsan analogs other than those specifically described herein also can be prepared by those skilled in the art by following methods known in the art as well as methods described herein.

Studies showing that exogenous fatty acids can be incorporated directly have been reported by Gorkovenko et al., Carbohydrate Polymers, 39: 79-84 (1999), the teachings of which are incorporated herein by reference in their entirety). Gorkovenko et al., (Carbohydrate Polymers 39: 79-84 (1999), the teachings of which are incorporated herein by reference in their entirety), used 0.5% (w/v) ¹³C₁-palmitic acid and 0.5% acetic acids as cosubstrates and reported an emulsan analog wherein GC-MS analysis of the 7475 and 87/88 mass ion pairs showed that 80% of emulsan C 16 fatty acid esters are incorporated intact from the C16 carbon source. Other studies have showed that the percent of C14, C15, C16, C17 and C18 fatty acid esters derived from the corresponding n-alkanoic acids that were mono unsaturated were 10.9, 25.6, 33.5, 69.7 and 84.7%, respectively (Gorkovenko et al., Proc. Am. Chem. Soc., Div. Polym. Sci. Eng. 72.92-93 (1995); Gorkovenko et al., Can. J Microbiol. 43:384-390 (1997); Zhang, et al., J. Biol. Macromol. 20: 9-21 (1997), the teachings of which are incorporated herein by reference in their entirety). An aerobic desaturation mechanism (Δ9 desaturase activity) has been reported for emulsan synthesis (Gorkovenko et al., Proc. Am. Chem. Soc., Div. Polym. Sci. Eng. 72:92-93 (1995); Gorkovenko et al., Can. J Microbiol. 43:384-390 (1997); Zhang, et al., J. Biol. Macromol. 20: 9-21 (1997), the teachings of which are incorporated herein by reference in their entirety). Unsaturated fatty acids of emulsan are believed to be directly synthesized from saturated fatty acids of a carbon source based on ¹³C₁-labeling experiments. Thus, it is likely that fatty acid supplements in A. calcoaceticus RAG-1 cultivations can be incorporated intact to large extents within chain lengths C15 to C18, and that the percent unsaturation is chain length dependent.

Generally, purified emulsan yields are from about 0.3 to about 1.8 g/L (Gorkovenko et al., Proc. Am. Chem. Soc., Div. Polym. Sci. Eng. 72:92-93 (1995)) (Gorkovenko et al., Can. J Microbiol. 43:384-390 (1997); (Zhang, et al., J. Biol. Macromol. 20: 9-21 (1997), the teachings of which are incorporated herein by reference in their entirety).

Emulsan and emulsan analogs employed in the methods and formulations of the invention can be characterized with respect to properties such as their colloidal properties as described, for example, by Gorkovenko et al., Can. J. Microbiol. 43:384-390 (1997) and Zhang, et al., J. Chem. Tech. and Biotech. 74:759-765 (1999), the teachings of which are incorporated herein by reference in their entirety. Structural changes in the emulsan polymer also can result in alteration in emulsification behavior and critical micelle concentration (CMC), as reported by Zhang, et al., J. Chem. Tech. and Biotech. 74:759-765 (1999), Surface tension has been reported to decrease with increasing emulsan concentration Zhang, et al., J. Chem. Tech. and Biotech. 74:759-765 (1999). For example, the CMCs ranged from about 25 to about 58 mg/L and surface tension and interfacial tension measurements have been reported to indicate stability between pH 2 and 10. Structural features are believed to influence emulsifying activity. For example, total fatty acid content and distribution of chain lengths have been reported to have significant influence on emulsification. Maximum emulsifying activity has been reported for emulsans containing about 400 nmol of total fatty acids per mg of emulsan (nmol/mg). Activity towards longer chain length oils has been reported to increase as the fatty acid side chain lengths increased. Thus, substrate-specific interactions between the emulsan fatty acid pendent groups and the dispersed phase have been suggested. A number of hexadecane-in-water emulsions prepared with droplet sizes between about 9 and about 19 μm have been reported to be stable with respect to coalescence for more than one year.

In one embodiment of the invention, emulsan and emulsan analogs are derived by growing A. calcoaceticus in ethylpropionate, myristic acid (C14:0) or ethanol and have the fatty acid contents shown in Table 1. As seen in Table 1 the carbon source affects the types and amounts of substituents on the polymer backbone. For example, the level of odd chain length fatty acid groups seen with ethylpropionate is about 12 mole percent 17-carbon chain length, while the use of myristic acid does not yield odd chain length pendant groups.

In another embodiment of the invention, emulsan analogs are obtained by employing saturated and unsaturated fatty acids with chain lengths of about C₁₁ to about C₁₈ as a carbon sources for A. calcoaceticus strain RAG-1 as discussed by Gorkovenko et al., Proc. Am. Chem. Soc., Div. Polym. Sci. Eng. 72:92-93 (1995), Gorkovenko et al., Can. J. Microbiol. 43:384-390 (1997); Zhang, et al., J. Biol. Macromol. 20: 9-21 (1997), the teachings of which are incorporated herein by reference in their entirety. Data characterizing emulsans obtained by feeding the bacteria n-alkanoic fatty acids of chain lengths 11:0(11 carbon atoms), 12:0(12 carbon atoms), 13:0(13 carbon atoms), 14:0 (14 carbon atoms), 15:0 (15 carbon atoms), 16:0 (16 carbon atoms), 17:0 (17 carbon atoms) and 18:0 (18 carbon atoms) as sole carbon sources are presented in Table 2. In general, chain lengths below about 15 carbons are found to have less of an influence on composition than those in the range of about 15-17. In the shorter chain length range it is believed that a high percentage of the fatty acids provided is directly incorporated onto the backbone. The mole percent of emulsan fatty acid esters having chain lengths equal to that of the C15, C16 and C17 n-alkanoic acid carbon sources have been reported as 53.1, 45.9 and 43.9%, respectively. In contrast, less apparent increases in C11, C12, and C13 emulsan fatty acids above that of a reference emulsan polymer generally are found in emulsans formed using the corresponding n-alkanoic acids as carbon sources.

In still another embodiment of the invention, emulsan analogs have the characteristics shown in Table 3. Table 3 illustrates the incorporation profiles with a series of 2-hydroxyl fatty acids [C 12:0(2-OH), C14:0(2-OH), C16:0(2-OH) and C18:0(2-OH] as sole carbon sources or when co-fed with myristic acid as described by Gorkovenko et al., Proc. Am. Chem. Soc., Div. Polym. Sci. Eng. 72:92-93 (1995), Gorkovenko et al., Can. J. Microbiol. 43:384-390 (1997), Gorkovenko, et al., Carbohydrate Polymers 39: 79-84 (1999), Zhang, et al., J. Biol. Macromol. 20: 9-21 (1997), the teachings of which are incorporated herein by reference in their entirety. Again, significant levels of modulation in the composition of the fatty acid pendent groups have been reported. For example, C12:0(2-OH) has been shown to increase to over about 64% of total fatty acids and 306 nmol/mg emulsan by providing this fatty acid as a carbon source. Significant quantities of 2-OH fatty acids with chain lengths ≧C14, up to 96 nmol/mg of emulsan or 15.2 mol % for C16:0(2-OH), have been also incorporated into emulsan by providing the corresponding 2-OH fatty acid carbon source. Furthermore, increasing the 2-OH fatty acid content in the medium has been shown to result in large has been shown to increase in total acid content for emulsans, up to about 955 nmol/mg of emulsan or about 23 wt %.

In a further embodiment, emulsan analogs can be prepared by depressing de novo fatty acid synthesis, as described by Gorkovenko et al., Proc. Am. Chem. Soc., Div. Polym. Sci. Eng. 72:92-93 (1995); Gorkovenko et al., Can. J. Microbiol. 43:384-390 (1997); Gorkovenko, et al., Carbohydrate Polymers, 39: 79-84 (1999); Zhang, et al., J. Biol. Macromol. 20: 9-21 (1997), the teachings of which are incorporated herein by reference in their entirety. Cerulenin, an irreversible inhibitor of β-ketoacyl-ACP synthase I and II activities, can be employed, for example at about 150 mg/L with ethanol (1% w/v) as the carbon source and palmitoleic acid (16:1, 9-cis) as the fatty acid supplement (0.2%). Following this procedure, the increase in the content of palmitoleic acid in emulsan due to the presence of cerulenin can be as high as 61% while in the control experiment, the content of 16:1 can be 19.9 mol % vs. 32.1 mole percent in the presence of cerulenin. Iodoacetamide (IAA, non-specific inhibitor of fatty acid metabolism) can also be employed to control the degree of substitution (d.s.). For example, at 0.5 mM of IAA the concentration of myristic acid in the product has been reported to increase from approximately 10 to about 30 mole percent.

In another embodiment of the invention, emulsan analogs are produced (e.g., secreted from) by mutants of A. calcoaceticus (also referred to herein as mutant A. calcoaceticus). In a particular embodiment, the mutant is a mutant of A. calcoaceticus RAG-1. In a preferred embodiment, the mutant is a transposon mutant of A. calcoaceticus RAG-1. In a more preferred embodiment, the mutant is a transposon Tn10 mutant of A. calcoaceticus RAG-1. The nucleic acid sequence of the Tn10 transposon and methods for using the Tn10 transposon for mutagenesis are known. (See, for example, Herrero, et al., J. Bacteriol., 172:6557-6567 (1990); Leahy, et al., J. Bacteriol., 175:1838-1840 (1993)).

A “mutant” refers to any A. calcoaceticus which differs from the nonmutant A. calcoaceticus, also referred to as native or wildtype bacterium, in a sequence of the genetic material of the bacterium. The mutant can be the result of a spontaneous mutation or the result of an experimental mutation (e.g., transposon mutagenesis). A mutant differs from the nonmutant by any change in any genetic sequence. For example, the difference in the gene sequence can be the result of a single or multiple base pair changes, an interruption in the genetic sequence, a frameshift mutation, or the random or selective insertion of an exogenous nucleic acid sequence (e.g., transposon, such as Tn10) into the genome of the bacterium. The mutant can have metabolic, physiology or phenotypic differences from the nonmutant which lead to the production and secretion of emulsan analogs.

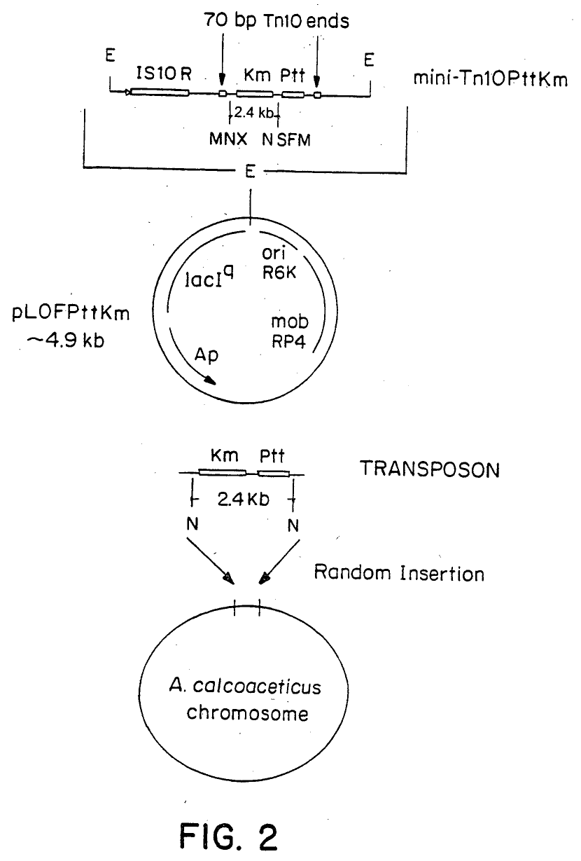

In a preferred embodiment, the mutant is a mutant produced by insertion of a transposon into the genome of the A. calcoaceticus bacterium (FIG. 2). In a preferred embodiment, transposon Tn10 is used to produce a Tn10 transposon mutant of A. calcoaceticus RAG-1 bacterium. Methods to produce mutants for use in the invention are well known to one of skill in the art. See, for example, Gutnick, D. L. et al., U.S. Pat. No. 4,311,829 (1982), the teachings of which are incorporated herein by reference in their entirety. For example, the nucleotide sequence and insertion strategies for the transposon Tn10 have previously been described. See, for example, Herrero, et al., J. Bacteriol. 172:6557-6567 (1990); Leahy et al., J. Bacteriol 175:1838-1840 (1993), the teachings of which are incorporated herein by reference in their entirety.

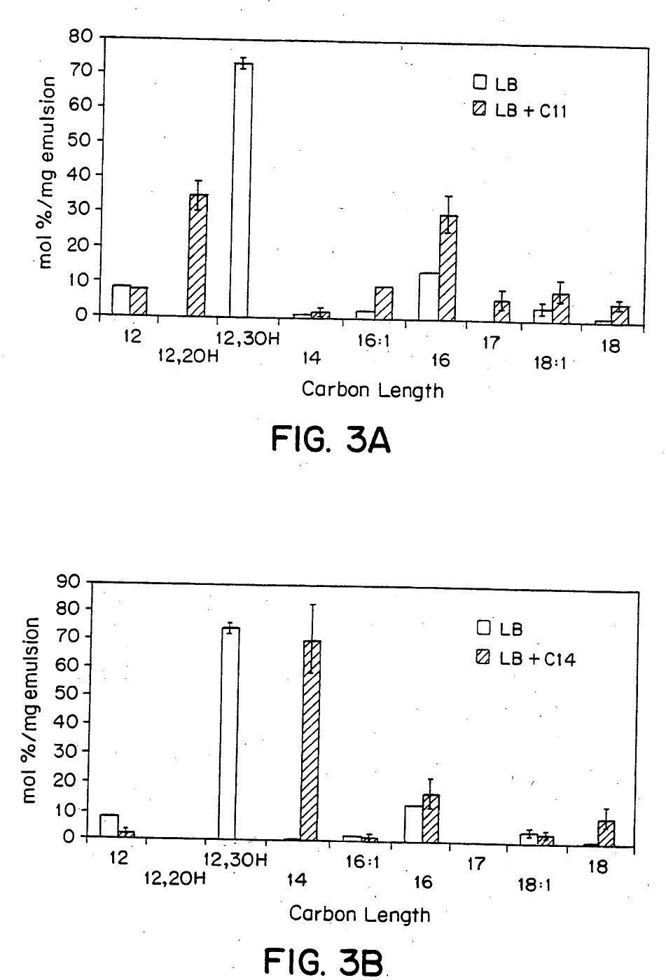

In another embodiment, the mutant bacterium can have a loss of β-oxidation pathways which promote direct incorporation of exogenous fatty acids. In yet another embodiment, the mutant bacterium can have a loss of fatty acid synthesis. Changes in P-oxidation pathways and fatty acid synthesis can be determined using standard techniques well known to one of skill in the art. In a further embodiment of the invention, emulsan analogs are produced from mutants grown on various carbon sources, such as, for example, the carbon sources described above. Different culture conditions can result in the production and secretion of emulsan analogs by mutants which differ in emulsification activity. For example, the emulsification activity and fatty acid profile of a Tn10 transposon mutant A. calcoaceticus RAG-1 varied when cultured in Luria Broth alone or Luria Broth containing varying fatty acids of varying carbon lengths (FIGS. 3A, 3B, 4A, 4B, 8).

The emulsan analogs of the invention produced and secreted by mutant bacterium can be combined with an antigen for use in the immunization formulations of the invention. One or more emulsan analogs from mutant bacterium can be mixed or administered with the antigen alone or with the antigen and an emulsan or emulsan secreted from a nonmutant bacterium. The terms nonmutant, wild type, native or control bacterium are used interchangeably herein to refer to a bacterium that is not a mutant bacterium.

The emulsan analogs of the invention produced and secreted by mutant bacterium can lead to immune responses (e.g., T-cell and/or B-cell activation and recruitment, antibody production to an antigen) which are similar to, less than or greater than emulsan analogs produced and secreted by nonmutant bacterium cultured under a variety of feeding strategies (e.g., varying fatty acid chain length, varying fatty acid density, fatty acids with varying amounts of saturated bonds).

It is envisioned that the mutants of the invention can be used to produce readily available cellular sources to generate large volumes of emulsan analogs for use in immunization formulations and methods of stimulating cytokine responses. Clonal populations of mutants can be stored, cultured and emulsan analogs with consistent properties generated under standards laboratory techniques. Mutants deficient in fatty acid utilization can lead to further ‘tailoring’ of structural profiles. Mutants can be grown on various carbon sources, such as the carbon sources described above, also resulting in further tailoring of emulsan analogs.

Thus, another aspect of the invention relates to a method of forming an emulsan analog composition for use as an adjuvant, comprising the steps of making a mutant bacterium, screening the mutant bacterium for the production and secretion of emulsan analogs, wherein the emulsan analog secreted from the mutant bacterium is used as an adjuvant or a stimulate.

In another embodiment of the invention, emulsan or emulsan analogs can include protein. In yet another embodiment, the protein present with emulsan or emulsan analog, when released from the cytoplasm or cell membrane of A. calcoaceticus, can be removed. Methods for removing the protein include but are not limited to hot phenol extraction and proteolytic digestion (e.g., proteinase K). “Deproteinized emulsan or deproteinized emulsan analog” refers to an emulsan or emulsan analog in which the protein has been removed, whereas “crude emulsan or crude emulsan analog” refers to an emulsan or emulsan analog in which the protein has not been removed.

The invention also relates to a method of stimulating cytokine release in a host comprising administering to the host an emulsan or an emulsan analog. The host can be a cell or a collection of cells (e.g., the macrophage cell line RAW264.7 (ATCC TIB-71) or peritoneal macrophages obtained from a host animal). The host can also be an animal host (e.g., mammal, avian, rodent). In a preferred embodiment, the host is a mammal (e.g., mouse, rat, guinea pig, sheep, goat, rabbit or primate such as a human). Organism is also used herein to refer to animal host. In a particular embodiment, the cytokine is TNF. Methods to assess cytokine release are well known to one of skill in the art. For example, commercially available ELISA (enzyme linked immunoabsorbent assay) kits can be purchased to determine the amount of TNF release (Genzyme Corp., Boston, Mass.).

It is also envisioned that the emulsan and emulsan analogs described herein will stimulate the production, release, activity or any combination thereof, of any cytokine, for example, Interleukin (IL)-10, IL-12, IL-9, Interferon-α, Interferon-β, Interferon-γ, Granulocyte-Colony-Stimulating Factor (GCSF), Epidermal Growth Factor (EGF), Leukemia Inhibitory Factor (LIF), Transforming Growth Factors (TGF) α and β, Vascular Endothelial Cell Growth Factor (VEGF), or Platelet-Derived Growth Factor (PDGF).

In a preferred embodiment, the cytokine release is accompanied by immunomodulation of the host. “Immunomodulation” of the host refers to any response of the host which leads to a modification or regulation of one or more immune responses. The immune responses can be, for example, the production of antibodies (e.g., IgG, IgM). Immunomodulation can also refer to the alteration of the immune system by the administration of the emulsan, emulsan analog alone or in combination with each other and an antigen that results in the binding of antibodies to the antigen to a bacteria, fungi, parasite, or virus or any antigenic portion thereof. As a consequence of the antibody binding to an antigen the host can survive a challenge to the native bacteria, fungi, parasite or virus. In this instance the immunomodulation of the host would provide prophylactic protection.

Thus, the immunization formulation comprising the antigen and emulsan or emulsan analog can be used as a vaccine. A vaccine refers to any immunization formula comprising an antigen and emulsan or emulsan analog described herein which result in immunological prophylaxis.

The emulsan, emulsan analog from nonmutant or mutant bacterium can be administered alone or simultaneously to a host with or without an antigen. Alternatively, or additionally the antigen and emulsan or emulsan analog can be administered separately to the host. For example, the host can be administered the antigen followed by administration of the emulsan or emulsan analog. Likewise, the host can be administered the emulsan or emulsan analog followed by administration of the antigen. One of skill in the art would be capable of determining a suitable administration strategy.

The emulsan, emulsan analog or immunization formulation of the invention can be administered to the host (e.g., mammal) using various routes of administration known in the art. Methods of administration of compositions for use in the invention include, but are not limited to, intradermal, intramuscular, intraperitoneal, intravenous, subcutaneous, intraocular, oral and intranasal. In a preferred embodiment, the route of administration is intramuscular or intraperitoneal. The emulsan, emulsan analogs and immunization formulations of the invention can also be administered to the host in combination with other physiologically acceptable medium (e.g., water, buffered saline, polyols such as glycerol, propylene glycol, liquid polyethylene glycol and dextrose solutions).

The amount, optimum concentration and dose of emulsan, emulsan analog, and antigen in the immunization formulation needed to elicit an immune response or immunodulation of the host can be determined empirically, according to procedures well known to one of skill in the art.

It is further envisioned that the immunization formulations and methods of stimulating cytokine release in a host can be accomplished employing lipopolysaccarides other than emulsan and emulsan analogs described herein.

The present invention is further illustrated by the following examples, which are not intended to be limiting in any way.

Exemplification

Experimental Methods

Emulsan Production and Purification

Growth of A. calcoaceticus: A. calcoaceticus RAG-1 and the transposon mutants were grown to early stationary phase in baffled Erlenmeyer flasks using gyrorotatory shaking (250 rpm) at 30° C. using established protocols (Shabtai, Y., Intl. J. Biol. Macromol. 12:145-152 (1990); Gorkovenko, A., et al., Proc. Am. Chem. Soc. Divi. Polym. Sci. Eng. 72:92-93 (1995); Gorkovenko, A., et al., Can J. Microbiol. 43:384-390 (1997), the teachings of which are incorporated herein by reference in their entirety). The cells were harvested by centrifugation at 6,000 rpm and the pellet resuspended in 1/20 the original volume using a 1:1 mixture of a 20% sterile aqueous glycerol solution and an equal volume of sterile minimal medium. The suspended cells were agitated and transferred in small aliquots to Eppendorf microfuges. The vials were stored at −70° C. until needed for inoculation of a medium. For inoculation of a polymer producing media (Gorkovenko, A., et al., Proc. Am. Chem. Soc. Div. Polym. Sci. Eng. 72:92-93 (1995), the teachings of which are incorporated herein by reference in their entirety), microfuge vials containing frozen stocks of A. calcoaceticus strains were removed from the cryogenic freezer and the contents thawed rapidly by agitation in a 37° C. water bath. A 200 μL volume of the microfuge tube contents per 50 mL culture volume was used to inoculate seed culture flasks. Seed cultures (3% v/v) taken at mid exponential phase were used for inoculation of flasks for polymer production.

Emulsan or Emulsan Analog Production: Emulsan and emulsan analog production was carried out as described previously (Gorkovenko, A., et al., Proc. Am. Chem. Soc. Div. Polym. Sci. Eng 72:92-93 (1995); Gorkovenko, A., et al., Can. J. Microbiol. 43:384-390 (1997); Zhang, J., et al., Intl. J. Biol. Macromol 20: 9-21(1997); the teachings of which are incorporated herein by reference in their entirety). Generally, about 0.3 to about 1.8 g/L of polymer were yielded from native, also referred to herein as nonmutant A. calcoaceticus strains, while transposon A. calcoaceticus mutants (also referred to herein as transposon mutants) were lower.

Fermentation experiments were carried out in baffled shake flasks. The choice of organism (native or mutant), and conditions, were based on the choice of structural analogs (chain length of predominant fatty acid side chains, unsaturation, hydroxylation) that were selected for the studies. The carbon source was included with or without additional carbon for metabolism (usually ethanol at 0.2%). In addition, ‘control’ compositions which were produced by growing A. calcoaceticus strain RAG-1 on ethanol or propionate were included in all procedures. A variety of emulsan analogs were generated from A. calcoaceticus RAG-1 as well as the transposon mutants by feeding fatty acids of different chain lengths (usually from about C11 to about C20 depending on the experiment—see Tables 1-4).

Emulsan Purification: The methods for emulsan purification were as previously published (Gorkovenko, A., et al., Proc. Am. Chem. Soc. Div. Polym. Sci. Eng 72:92-93 (1995); Gorkovenko, A., et al., Can. J. Microbiol. 43:384-390 (1997); Kaplan, N., et al., Appl. Environ. Microbiol. 44(6):1335-1341(1982), the teachings of which are incorporated herein by reference in their entirety). Cell cultures were harvested by centrifugation and the polymer was precipitated from solution by the addition of ammonium sulfate to about 40% saturation while the solution was maintained at 4° C. The precipitated product was isolated by centrifugation, desalted by dialysis, concentrated by tangential flow filtration and lyophilized. Residual aliphatic impurities were removed from the product by Soxhlet extraction with ether. All of the polymers were treated to remove associated protein using a hot phenol extraction method (Rosenberg, E., et al., Appl. Env. Microbiol., 37:409-413 (1979), the teachings of which are incorporated herein by reference in their entirety).

Structural Analysis

Gas Chromatography (GC) Mass Spectroscopy (MS) and Flame Ionization Detection (FID) Analysis of Side Chains: Emulsans were analyzed by GC-MS to establish the composition of the lipid product fraction as well as the degree of substitution (d.s. nmole of lipid/mg of product). Standards were used to confirm mass spectra of the fatty acids and to obtain relative response factors for quantitative analyses where tetradecane serves as the internal standard. For determination of the fatty acid moieties present on the polysaccharide backbone, emulsans were cleaved and transesterified in the presence of methanol and H₂SO₄ to yield the corresponding methyl esters. The products (about 20 to about 50 mg) were dissolved in a mixture of 2 mL of 1% H₂SO₄ in methanol and 1 mL of toluene containing 2 g/L tetradecane as internal standard. The mixture was heated for 1 h at 100° C. in sealed vials under argon. A Hewlett Packard Model 5890 Series 2 gas spectrometer equipped with a mass selective detector (model 5971) and an HP Ultra 2 (5% Phenlyl Methyl Silicone) capillary column (Hewlett Packard, 25 m×0.25 mm, 0.33 mm film thickness) was used for component lipid structural analyses.

Functional Analysis

Measurements of Surface and Interfacial Tension of Solutions, Critical Micelle Concentration (CMC), and Stability of Emulsions of Structural Analogs: An American Society for Testing and Materials standard method (ASTM D 1331-89) and a du Nouy interfacial tensiometer equipped with 6 cm platinum-iridium ring were used for determinations of surface and interfacial tension and CMC of the different emulsans used in the bioassays.

N-Alkanes (C8-C18) were used for interfacial tension measurements. CMC values were obtained from the dependence of surface tension on solution surfactant concentration. In addition, emulsions formed with a high shear homogenizer (13,500s⁻¹) were prepared and analyzed using a Horiba LA-910 laser light scattering instrument. The system consisted of 6.25% hexadecane by volume with an emulsan concentration of 3.9 gdm-3 oil phase. The samples were gently shaken prior to their introduction into the Horiba sample chamber to redisperse the creamed oil phase and samples can be also diluted as needed. These methods are described by Zhang, J., et al., J. Chemical Technology Biotechnology 74.759-765 (1999), the teachings of which are incorporated herein by reference in their entirety.

Measurements of Emulsification Properties: Emulsification activity was determined based on a modification of a literature method described by Rosenberg, E., et al., Appl. Env. Microbiol. 37:409-413 (1979), the teachings of which are incorporated herein by reference in their entirety. A 0.1 to 0.5 ml aqueous solution of the emulsan analog was mixed with 0.1 ml of a standard substrate (usually use about 1:1 hexadecane/2-methylnaphthalene). The mixture was introduced into a 100 ml flask with Tris-magnesium buffer to a final volume of 7.5 ml. The assay mixture was incubated at 30° C. with reciprocal shaking (150 strokes per min) for 1 hr. The turbidity of the assay mixture was assayed by a Klett-Summerson photometer (green light). One unit of emulsifying activity per ml is defined as the concentration of a polymer solution which yields 100 Klett units (K.U.) in the assay mixture.

Macrophage Stimulation Assays

Sources of Macrophages: Murine macrophage cell line, RAW 264.7 cells (ATCC No.TIB-71) were used for the activation screens. RAW 264.7 cells were maintained and sub-cultured as suggested at ATCC. Briefly, cells were maintained in DMEM containing 10% FCS at 37° C., 5% CO₂. Every fourth day the cells were split 1:6 by scraping and centrifugation at 500×g. The cells were resuspended in fresh media and placed in fresh T-flasks.

Samples of emulsan were also tested on resident peritoneal macrophages from BALB/c mice as well as the LPS-responsive and non-responsive macrophage cell lines HeNC2 and GG2EE. Peritoneal macrophages were obtained from a sterile lavage after euthanasia.

Stimulation of TNF production: RAW 264.7 cells were plated at 8×10⁴ cells/well in DMEM media with 10% FCS in 96-well culture plates. After 48 hours of incubation at 37° C., media were replaced with fresh DMEM without sera, and emulsan analogs to be tested were then added to the cells at appropriate concentrations, with triplicate wells for each test. Cells were incubated 18-20 hours, and supernatants were then collected for subsequent assay. Lipopolysaccaride (LPS) at 100 ng/ml served as a positive control for macrophage stimulation. Lipopolisaccharides, abbreviated herein as LPS, are a major constituent of the cell wall of gram-negative bacteria and includes a lipid A protein attached to a polysaccharide chain. LPS extracted from various serotypes of E. coli, Salmonella enteritidis and from other gram negative bacteria are commercially available. The LPS employed in these experiments was obtained from Escherichia coli Serotype 055:B5.

Resident peritoneal macrophage cells from a sterile lavage were plated at 2×10⁵ cells/well in RPMI-1640 media with 5% FCS, and 5 μg/ml Polymyxin B (to control for LPS contamination) in flat-bottomed 96-well tissue culture plates. After 1-3 hours incubation at 37° C., media were replaced with RPMI containing 2 μg/ml indomethacin, and incubated 30 minutes. Emulsan variants to be tested were then added to the macrophages at appropriate concentrations, with triplicate wells for each test. Cells were then incubated for another 18-20 hours, and supernatants were then collected for subsequent assay.

HeNC2 and GG2EE cells (Nathan et al., Cell 88:417-426 (1997)) were plated at 1×10⁵ cells per well in a 96-well tissue culture plate in 100 μl of RPMI-1640 with 10% FCS. Stimulants (i.e., emulsans) were diluted in RPMI/10% FCS and added to the cells to a final volume of 2001. After 24 hours of stimulation, the plates were spun down at 500×g for 10 minutes to pellet the cells. Culture supernatants were removed and tested as above. The supernatants for these macrophage cultures were assayed for TNF by sandwich ELISA (Duo-Set, Genzyme, Corp., Boston, Mass.).

Cytokine Determination

Cytokine release was quantified by a sandwich ELISA according to the manufacturer's instructions (Genzyme), with slight modifications as noted below. Briefly, Nunc Maxisorp 96-well plates were coated overnight at 4° C. with 6 μg/ml of capture antibody (goat anti-murine TNF). Plates were washed with PBS/Tween-20 three times, and blocked with 1% BSA in PBS. After two hours incubation at 37° C., the plates were washed again, and 50 μl of wash buffer added. Standards and macrophage supernatants were added (50 μl) to the plates and incubated overnight at 4° C. Plates were washed, and scoring antibody (hamster anti-murine TNF conjugated to horseradish peroxidase) was added at 3 μg/ml dilution in 1% BSA in PBS/Tween-20. Plates were incubated for two hours at 37° C. and then washed. TMB substrate (Sigma Chemical Co., St. Louis, Mo.) was added to each well and color allowed to develop for 10-30 minutes. Color development was stopped by the addition of 2N H₂SO₄, and plates were read at 450 nm. TNF released was determined by a standard curve based on recombinant murine TNF at several concentrations.

Adiuvanticity of Structural Analogs including Isotype Specific Responses/Preliminary Toxicity

Immunization Protocol: The adjuvant activity of emulsan was assayed using a classical hapten-carrier immunization protocol. (Coligan, et al., “Current Protocols in Immunology” John Wiley & Sons, (1991)). These assays were performed with the ‘native’ proteinated and deproteinated emulsan that had been produced from cultures grown on ethanol as a carbon source.

Forty 6-8 week-old female BALB/c mice were randomly placed in eight groups of five mice and immunized (Table 5). Pre-immune sera were obtained 3 days prior to primary immunization. Antigen (dinitrophenol coupled to keyhole limpet hemocyanin referred to as DNP-KLH and adjuvant were mixed by repeated aspiration through an 18-gage needle. Each mouse was immunized intraperitoneally (i.p.) with 200 μl total volume of adjuvant (crude or deproteinized emulsan; or Freund's adjuvant) and antigen. Mice were boosted after 28 days, and sera were taken every 3 days after boost until day 21 post-boost, and then again at 6 weeks and 9 weeks. Total DNP-specific antibody titers was determined by ELISA. Controls included injection of mice with emulsan alone in the absence of antigen.

ELISA Assay: Briefly, flat-bottomed 96-well plates (Nunc Maxisorp) were coated overnight at 4° C. with 5 μg/ml BSA-DNP in 0.05 M carbonate buffer, pH 9.5. After the overnight incubation, plates were washed three times with wash buffer (PBS/Tween-20, 0.05%), and then blocked for 2 hours at room temperature with 1% BSA in PBS. Plates were again washed three times, and 501 μl of wash buffer was added to each well. Sera from tail bleeds were diluted 1:100, and then 5-fold serial dilutions of the 1:100 stocks were made. The antiserum dilutions were added to the plates (50 μl) in duplicate and incubated overnight at 4° C. After three washes, 100 μl of HRP-conjugated goat-anti-mouse IgG (H+L, Bio-Rad) diluted 1:10,000 in 1% BSA in PBS/Tween-20 (0.05%) was added to each well and incubated 2 hours at room temperature. Plates were again washed three times, and 100 μl of TMB substrate (Sigma) was added to each well. Color was allowed to develop for 10 or 20 minutes, when 2N H₂SO₄ was added to stop the reaction. Absorbance was read at 450 nm.

Histological Analysis—Toxicity: An examination of gross pathology was performed, and tissue sections from spleen, liver (with gall bladder), lung (inflated with formalin), kidney, heart, injection site and draining lymph nodes were prepared and examined for signs of inflammation or necrosis.

Mutants and Selection

Generation of A. calcoaceticus RAG-1 Transposon Mutants: Transposon mutants of A. calcoaceticus RAG-1 (ATCC 31012) were generated using a modified Tn10 transposon carrying a kanamycin resistance gene (mini-Tn10PttKm) (Herrero et al., J. Bacteriol, 172: 6557-6567 (1990), the teachings of which are incorporated herein by reference in their entirety, using previously described procedures (Leahy et al., J. Bacteriol, 175:1838-1840 (1993), the teachings of which are incorporated herein by reference in their entirety). Conjugation using Escherichia coli SM10 as donor was found to be more effective than electroporation to transform A. calcoaceticus RAG-1 directly with the isolated plasmid pLOFPttKm containing the transposon mini-Tn10PttKm. After IPTG induction of the transposase encoded on the plasmid, antibiotic selection with chloramphenicol and kanamycin was used to isolate A. calcoaceticus RAG-1 transconjugants containing chromosomal mini-Tn10PttKm insertions after IPTG induction of the transposase encoded on the plasmid. Individual transposon mutants were selected and propagated in LB broth supplemented with kanamycin.

Screening of Transposon Mutants: Transposon mutant selection were selected on the basis of metabolic deficiencies, particularly in fatty acid metabolism, which can be used to promote direct incorporation of exogenous fatty acids. For example, transposon mutants were selected based on two phenotypic functions: (a) loss of β-oxidation pathways (to promote direct incorporation of exogenous fatty acids), (2) loss of fatty acids synthesis. None of the specific enzymes or encoding genes responsible for these structural feathers of emulsan have been characterized in A. calcoaceticus RAG-1. Fatty acid incorporation by a series of the above transposon mutants suggests additional options in the control of the structural features of these lipopolysaccharides. For example, lower levels of fatty acid substitution is a general characteristic of these mutants. Some of these data are provided in Table 4.

One selected, colonies of transposon mutants were replica plated from LB agar, a nutritionally complex medium) onto minimal medium (Shabtai, et al., J. Bacteriol. 161:1176-1181 (1985), the teachings of which are incorporated herein by reference in their entirety), agar plates supplemented with ethanol (1% v/v) or fatty acid (oleic acid 1% w/v) as a sole carbon source. Mutants capable of growth on fatty acids but not in ethanol as a sole carbon source can block fatty acid biosynthesis and mutants deficient in β-oxidation pathways would be incapable of growth on fatty acids but can grow on ethanol. Additional nutritional auxotrophs can be incapable of growth on either minimal medium.

Molecular Characterization of Transposon Mutants: Standard molecular biology techniques (Ausubel et al., “Current Protocols in Molecular Biology” John Wiley & Sons, (1995), the teachings of which are incorporated herein by reference in their entirety), were used to characterize and isolate the genomic DNA from transposon mutants of A. calcoaceticus RAG-1. Kanamycin resistant clones, carrying the mini-Tn10PttKm cassette and surrounding genomic DNA were selected. The sequence of DNA of the transposon mutant surrounding the transposon cassette was obtained using routine cloning and sequencing methods such as restriction enzyme digestion and PCR based nucleic acid sequencing with the oligonucleotide primers, to conserved regions of the transposon, such as 5′-GGA CGG CGG CTT TGT TG-3′ (SEQ ID. NO.: 1) and 5′-CCT CGG TGG CAC GGC GGA TGT-3′ (SEQ ID. NO.: 2).

Storage of Transposon Mutant Strains: Mutants were stored at −80° C. in liquid growth media after the addition of an equal volume of sterile 40% (v/v) glycerol.

Results

Structural ‘Tailoring’ of Emulsan Synthesized by A. calcoaceticus RAG1

The metabolic flexibility of the bacterium to incorporate exogenous fatty acids under a variety of culture strategies was explored. Significant manipulation in both the composition and degree of fatty acid substitution could be achieved. Saturated fatty acids with chain lengths of about C11 to about C18 were studied as sole carbon sources for A. calcoaceticus strain RAG-1. Some of the data from these studies are presented in Tables 2-4. Initially, emulsans synthesized either with ethylpropionate, myristic acid (C14:0) or ethanol were studied (Table 2). The study illustrates two important points: first, the structural complexity of emulsan with a diverse set of fatty acid substituents on the polysaccharide backbone; second, there is a clear influence of carbon source on the types and amounts of substituents. One clear indication of this effect is the significant level of odd chain length fatty acid groups (about 12 mole percent 17-carbon chain length when ethylpropionate is used), while the use of myristic acid did not yield odd chain length pendant groups. In general, purified emulsan yields are from about 0.3 to about 1.8 g/L.

In a second series of studies, n-alkanoic fatty acids of chain lengths 11:0, 12:0, 13:0, 14:0, 15:0, 16:0, 17:0 and 18:0 were used as sole carbon sources to explore the impact on emulsan production levels and structural features. In general, chain lengths below 15 carbons had less of an influence on composition than those in the range of 15-17. These results are illustrated in Table 2. In this range a high percentage of the fatty acids provided were directly incorporated onto the backbone. The mole percent of emulsan fatty acid esters having chain lengths equal to that of the C15, C16 and C17 n-alkanoic acid carbon sources were 53.1, 27.2 and 43.9%, respectively. In contrast, less apparent increases in C11, C12, and C13 emulsan fatty acids above that of a reference emulsan polymer were found in emulsans formed using the corresponding n-alkanoic acids as carbon sources.

TABLE 1
Fatty acid composition of emulsan produced by
A. calcoaceticus grown on ethyl propionate,
myristic acid (C14) or ethanol, numbers in columns
are mmol % with nmol/mg in parenthesis).

| Fatty Acid Content | Ethyl Propionate | Myristic Acid | Ethanol |
|---|---|---|---|
| 10:0 | 0 | 0.9(4) | 2.6(11) |
| 12:0 | 12(50) | 15(57) | 7(32) |
| 13:0 | 2(9) | 0 | 4(17) |
| 12:0, 2-OH | 5(21) | 9(33) | 14(59) |
| 12:0, 3-OH | 25(104) | 15(56) | 13(56) |
| 14:1 | 0 | 2(6) | nd |
| 14:0 | 0.9(4) | 12(47) | 3(14) |
| 15:1 | 0 | 0 | 1(2) |
| 16:1 | 8(33) | 18(69) | 8.2(36) |
| 16:0 | 13(56) | 18(68) | 23(99) |
| 17:1 | 5(23) | 0 | 0.3(1.4) |
| 17:0 | 7(27) | 0 | 0.4(2) |
| 18:1 | 15(64) | 8(30) | 13(56) |
| 18:0 | 2(10) | 1(4) | 5(20) |
| Unidentified | 3(11) | 2(12) | 1(5) |
| Totals | 100(420) | 100(386) | 100(436) |

To validate that the exogenous fatty acids were incorporation directly, 0.5% (w/v) ¹³C₁-palmitic acid and 0.5% acetic acids were used as cosubstrates. An emulsan was formed wherein GC-MS analysis of the 74/75 and 87/88 mass ion pairs showed that 80% of emulsan C16 fatty acid esters were incorporated intact from the C16 carbon source. In addition, an aerobic desaturation mechanism (Δ9 desaturase activity) was established for emulsan synthesis. Unsaturated fatty acids of emulsan were directly synthesized from saturated fatty acids of a carbon source based on ¹³C₁-labeling experiments. Thus, that fatty acid supplements in A. calcoaceticus RAG-1 cultivations can be incorporated intact to large extents within chain lengths C15 to C18, and the percent unsaturation is chain length dependent. In particular, emulsan compositions were found that showed significantly improved emulsification activity (by a factor of 3) and this was directly related to the side chain composition. Specifically, enhanced incorporation of long side chain groups gave polymers with improved emulsification activity on longer chain n-alkane substrates.

TABLE 2
Some examples of the influence of fatty acid chain
lengths from 15 to 18 carbons as carbon source feed on the
fatty acid profiles of emulsans synthesized by A. calcoaceticus.
Emulsan Fatty Acid Composition (nmol/mg
emulsan (% wt/wt)^{b}

| Fatty | | | | | |
|---|---|---|---|---|---|
| Acid | 15:0 + | | | 18:0 + | Total Fatty |
| Source* | 15:1 | 16:0 + 16:1 | 17:0 + 17:1 | 18:1 | Acids |
| 15:0 | 217(5.2) | 43(1.1) | 25(0.7) | 29(0.8) | 408(9.8) |
| 16:0 | — | 299(6.8) | — | 18(0.5) | 651(13.8) |
| 17:0 | 25.(0.6) | 14(0.3) | 184(4.9) | 9(0.3) | 418(9.7) |
| 15:0 + | 119(2.9) | 17(0.4) | 229(6.2) | 10(0.3) | 528(12.8) |
| 17:0^{c} | | | | | |
| 18:0 | — | 105(2.7) | — | 52(1.5) | 555(12.2) |

*The carbon source concentration in the growth medium was 1% wt/vol.
^{b}wt/wt = 100 X (total fatty acid weight/emulsan weight).
^{c}A 1:1 mol/mol mixture was used.

Additional studies were conducted to explore incorporation profiles with a series of 2-hydroxyl fatty acids [C12:0(2-OH), C14:0(2-OH), C16:0(2-OH) and C18:0(2-OH] as sole carbon sources or when co-fed with myristic acid (Table 3). Again, significant levels of modulation in the composition of the fatty acid pendent groups were observed. For example, C12:0(2-OH) increased to over 64% of total fatty acids and 306 nmol/mg emulsan by providing this fatty acid as a carbon source. Significant quantities of 2-OH fatty acids with chain lengths about ≧C14, up to about 96 nmol/mg of emulsan or about 15.2 mol % for C 16:0(2-OH), were also incorporated into emulsan by providing the corresponding 2-OH fatty acid carbon source. Furthermore, increasing the 2-OH fatty acid content in the medium resulted in large increases in total fatty acid content for emulsans, up to about 955 nmol/mg of emulsan or about 23 wt %.

Depression of de novo fatty acid synthesis so that increased incorporation of exogeneous fatty acids was achieved was also investigated. Cerulenin, an irreversible inhibitor of β-ketoacyl-ACP synthase I and II activities, was studied at 150 mg/L with ethanol (1% w/v) as the carbon source and palmitoleic acid (16:1, 9-cis) as the fatty acid supplement (0.2%). The increase in the content of palmitoleic acid in emulsan due to the presence of cerulenin was dramatic, 61%. In the control experiment, the content of 16:1 was 19.9 mol % vs. 32.1 mole percent in the presence of cerulinin. The influence of iodacetamide (IAA, non-specific inhibitor of fatty acid metabolism) on emulsan fatty acid composition was evaluated using myristic acid as the sole carbon source and cosubstrate mixtures of myristic and acetic acids. At 0.5 mM of IAA the concentration of myristic acid in the product was increased from about 10 to about 30 mole percent. Moreover, the use of these inhibitors enabled control of the degree of substitution (d.s.) by a factor of about 2.

TABLE 3
Incorporation of 20 hydroxy fatty acids in emulsans provided
at a total of 1% (wt/vol).

| | | | ^{b}Odd Chain | Odd Chain | ^{c}Total Fatty |
|---|---|---|---|---|---|
| | | ^{a}2-OH incorp. | Fatty Acids | Fatty Acids | Acids, wt % |
| Fatty Acid | C14:0(2-OH) | (mol %), nmol/mg | mol %, | mol %, | (nmol/mg |
| Source | ratio g/100 ml | emulsan | nmol/mg n − 1 | nmol/mg, total | emulsan) |
| C14 | 1.0/0.0 | — | 0 | 0 | 9.3(399) |
| C14:0/C12:0 | 0.5/0.5 | 27.4(101) | 0 | 1.7(6) | 8.5(370) |
| (2-OH) | | | | | |
| C14:0/C14:0 | 0.5/0.5 | 5.9(16) | 1.1(3) | 4.8(13) | 6.3(261) |
| (2-OH) | | | | | |
| C14:0/C16:0 | 0.5/0.5 | 2.9(10) | 21.5(77) | 24.8(88.6) | 8.2(359) |
| (2-OH) | | | | | |
| C14:0/C18:0 | 0.5/0.5 | 3.6(22) | 21.0(132) | 24.7(155) | 14.9(628) |
| (2-OH) | | | | | |
| C14:0/C12:0 | 0.25/0.75 | 64.4(306) | 0 | 3.8(18) | 10.7(476) |
| (2-OH) | | | | | |
| C14:0/C14:0 | 0.25/0.75 | 11.0(59) | 2.2(12) | 11.1(60) | 12.9(540) |
| (2-OH) | | | | | |
| C14:0/C16:0 | 0.25/0.75 | 15.2(96) | 15.1(95) | 10\9.1(120) | 14.9(632) |
| (2-OH) | | | | | |
| C14:0/C18:0 | 0.25/0.75 | 1.4(14) | 27.6(264) | 31.9(305) | 23.0(955) |
| (2-OH) | | | | | |
| C12:0 | 0/1.0 | — | — | — | — |
| (2-OH) | | | | | |
| C14:0 | 0/1.0 | 6.6(31) | 3.7(17) | 20.8(97) | 11.2(462) |
| (2-OH) | | | | | |
| C16:0 | 0/1.0 | 2.9(18) | 29.4(184) | 38.1(239) | 14.7(626) |
| (2-OH) | | | | | |
| C18:0 | 0/1.0 | 7.3(57) | 32.2(252) | 38.3(300) | 19.0(783) |
| (2-OH) | | | | | |

^{a}Emulsan 2-OH fatty acid substituents with chains lengths equal to the 2-OH fatty acid carbon source.
^{b}Emulsion side chains with one carbon shorter than the added 2-OH fatty acid carbon source.
^{b}Percentage by weight of total fatty acids in the emulsan produced.

Transposon Mutants

Several nutritional auxotrophs (13D, 52D, and 62C) capable of growth on LB (a nutrient rich medium) but not on minimal medium, and a mutant blocked in fatty acid biosynthesis (VRBS-1), capable of growth on minimal medium with fatty acid (but not ethanol) as a sole carbon source, were chosen for further emulsan characterization. An additional mutant (FF8) which exhibited a mucoid colony morphology, suggesting altered extracellular polysaccharide expression, was also identified.

Genomic DNA fragments containing the mini-Tn10PttKm cassette from mutants 52D and VRBS 1 were cloned into E. coli and the sequence of the surrounding DNA from each insertion obtained by primer walking from sequences conserved in the transposon. The gene interrupted in mutant 52D encoded a protein showing high homology to CysI, part of a sulfite reductase holoenzyme involved in cysteine biosynthesis. Supplementation of minimal medium with cysteine restored growth of mutant 52D on both ethanol and fatty acids as sole carbon sources. Fatty acid biosynthetic mutant VRBS-1 was disrupted in the biotin synthase gene bioB, and growth of the mutant on ethanol as a sole carbon source was rescued by supplementation of the medium with biotin.

Emulsan Analogs from Transposon Mutants

The analysis of fatty acid incorporation by a series of transposon mutants suggests additional options in the control of the structural features of these lipopolysaccharides. For example, lower levels of fatty acid substitution is a general characteristic of these mutants. As described later, this has important implications in the nature of the cytokine response. Some of the data characterizing these different emulsans are provided in Table 4.

Fatty acid profilers of Mutant 2 emulsan analog (M2) produced from Luria Broth alone and Luria Broth with undecanoic acid (C11 1% w/v) as a carbon source are shown in FIG. 3A. The total nmoles of fatty acids per milligram of emulsan were 33 and 10 for transposon mutant 2 when cultured in the presence of Luria Broth and Luria Broth with C11, respectively.

Fatty acid profile (mole %/mg emulsan of transposon mutant 2 (M2) cultured in the presence of Luria Broth alone and Luria Broth with myristic acid (C14 1% w/v) as a carbon source are shown in FIG. 3B. The total nmoles of fatty acids per milligram of emulsan were 33 and 8 for transposon mutant 2 when cultured in the presence of Luria Broth and Luria Broth with C14, respectively.

TABLE 4
Incorporation of fatty acids into emulsan by selected transposon
mutants of A. calcoaceticus.

| | | | | Emulsi- | Emulsi- |
|---|---|---|---|---|---|
| | | | Total Fatty | fication | fication |
| | | Emulsan | Acids | Activity | Activity |
| Transposn | Carbon | Yield | (nmol/mg | on | on |
| Mutant | Source | 6-days, g/L | emulsan) | C16 | C12 |
| control | Ethyl | 1.0 | 400 | 100 | — |
| | propionate | | | | |
| control | Ethanol | 1.0 | 170 | 190 | 48 |
| control | LB Broth | 0.3 | 76 | 10 | 5 |
| 52D (aux)^{a} | LB Broth | 0.2 | 80 | 18 | 18 |
| 52D (aux) | LB + C14 | 0.7 | 123 | 343 | 163 |
| 13D (aux) | LB Broth | 0.2 | 230 | 5 | 7 |
| 13D (aux) | LB + C14 | 0.4 | 107 | 6 | 6 |
| 62C (aux) | LB Broth | 0.2 | 175 | 30 | 36 |
| 62C (aux) | LB + C14 | 0.4 | 85 | 2 | 6 |
| VRBS-1^{b} | LB Broth | 0.5 | ND | 15 | ND |

^{a}aux = nutritional auxotroph
^{b}mutant deficient fatty acid biosynthesis

Protein Content and Removal

The protein present with emulsan once it is released from the cell membrane by A. calcoaceticus can be efficiently removed either by hot phenol extraction or by proteolytic digestion. Protein content in weight percent of emulsan was 16.4% (native emulsan—produced on palmitate), 0.7% after hot phenol extraction, and 0.8% after treatment with proteinase K. Emulsification activity (Klett Units) was 229±3 for the native emulsan, 175±5 for the hot phenol extracted emulsan, and 187±7 for the proteinase K treated material. Surface tension data in dynes/cm were 60.6, 69.3 and 67.5, respectively. Emulsification assays were run with 0.15% hexadecane. The content of fatty acids, in terms of nmol of fatty acid per mg of emulsan, remained in the 410 to 470 range for all three samples. Thus, either hot phenol extraction or proteolytic digestion for protein removal from emulan or emulsan analogs is suitable, although all other samples prepared for subsequent in vitro or in vivo studies were with hot phenol to avoid potential contamination with the enzyme used in the hydrolytic reaction.

Emulsification Properties of Emulsan

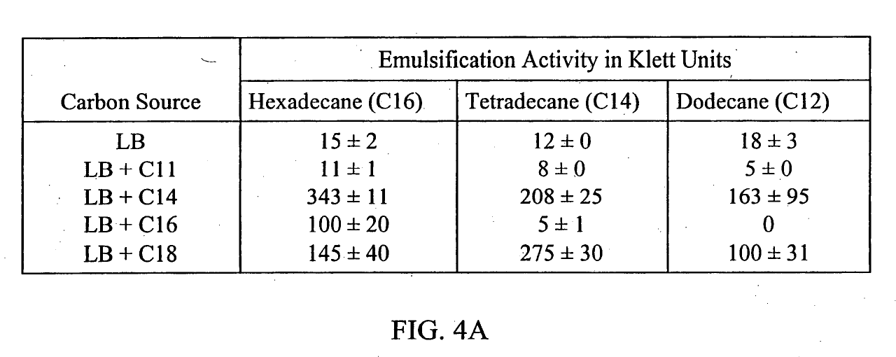

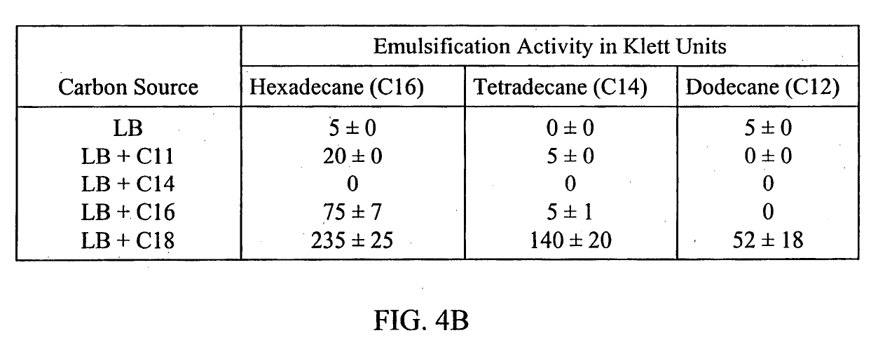

To determine whether structural changes in the emulsan resulted in alteration in emulsification behavior and critical micelle concentration (CMC), the colloidal properties of the emulsan were determined. Surface tension decreased with increasing emulsan concentration, as expected for typical surfactants. The CMCs ranged from about 25 to about 58 mg/L and surface tension and interfacial tension measurements indicated stability between pH 2 and 10. Structural features were found to influence emulsifying activity. For example, total fatty acid content and distribution of chain lengths had significant influence. Maximum emulsifying activity was found for emulsans containing about 400 nmol of total fatty acids per mg of emulsan (nmol/mg) and activity towards longer chain length oils increased as the fatty acid side chain lengths increased as well. Thus, substrate-specific interactions between the emulsan fatty acid pendent groups and the dispersed phase are suggested. It is also important to note that the hexadecane-in-water emulsions were prepared with droplet sizes between about 9 and about 19 μm and many of these emulsions were stable with respect to coalescence for months. Emulsification activity of transposon mutant 1 grown in Luria Broth (LB) alone and Luria Broth with fatty acid of varying carbon lengths (C11, C14, C16, C18) are shown in FIG. 4A and emulsification properties of transposon mutant 2 grown in the presence of Luria Broth (LB) alone and Luria Broth with fatty acid of varying carbon lengths (C11, C14, C16, C18) are shown in FIG. 4B.

Stimulation of Murine Macrophages by Emulsan

The macrophage response to emulsan produced on an ethanol feed source was assayed. The effects with and without the bound protein were determined. Furthermore, removal of the protein permitted a determination that immunomodulatory properties of the polymer were specific for the lipopolysaccharide, not a result of contaminating protein and that the absence of protein did not affect the emulsification properties of the polymer.

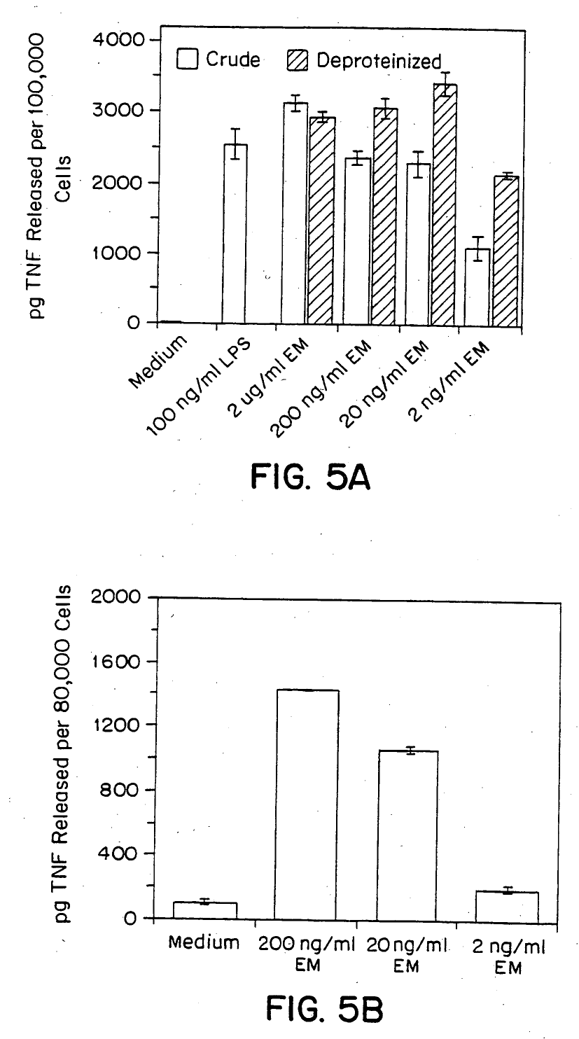

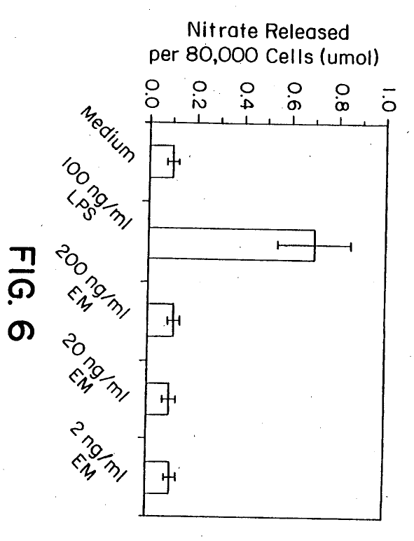

The immunomodulatory effects of emulsan or deproteinized emulsan in macrophages release TNF were determined. The results show that for proteinated and deproteinated emulsan stimulate TNF release (FIG. 5A). FIGS. 5A and 5B illustrate release of TNF by murine primary macrophages and RAW 264.7 cells, stimulated in culture with emulsans at various concentrations. The results show a dose-dependent release of TNF in response to emulsan stimulation from both macrophage sources. The response is approximately 20-fold lower than that of LPS on a per weight basis. Nitrite was not release in response to emulsan, as shown in FIG. 6.

Emulsan is a bacterial product, and because of the possibility of LPS contamination, macrophage activation assays on macrophage cell lines derived from the LPS-responsive and LPS non-responsive mice, C3H/HeJ and C3H/FeJ mice were performed. Methods for the production and maintenance of cell lines HeNC2 (LPS-responsive) and GG2EE (LPS-non-responsive) are known in the art (See, for example, Nathan et al., Cell 88:417-426 (1997)). The HeNC2 and GG2EE cells were stimulated with emulsan, and three structural analogs of sophorolipid (SL) for comparison.

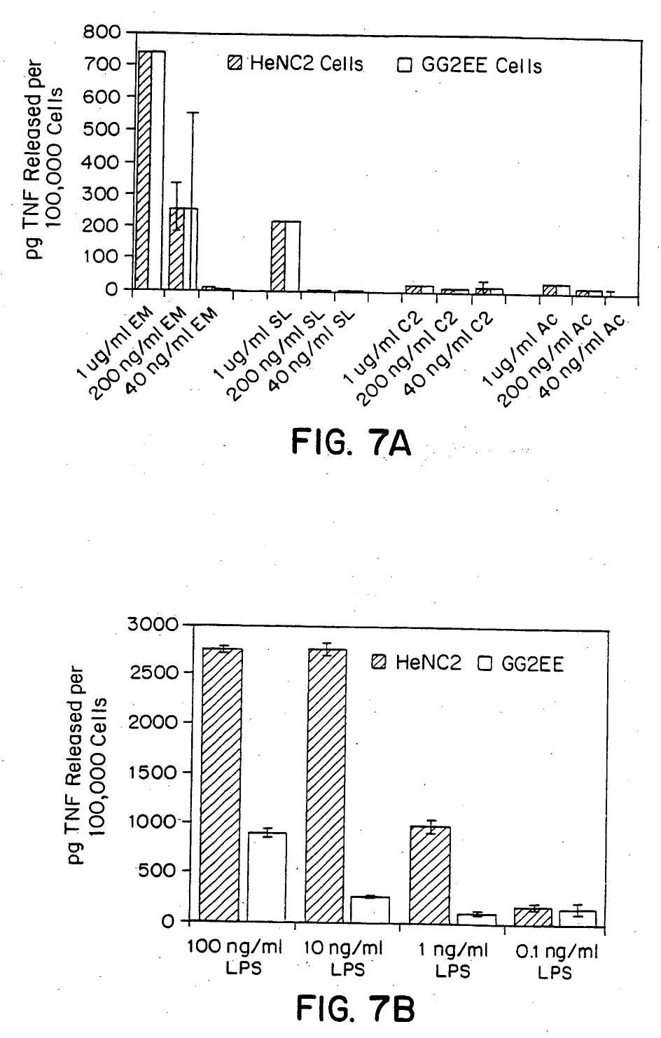

FIGS. 7A and 7B demonstrates the LPS-independent manner in which emulsan induces TNF. Emulsan, sophorolipid, and LPS were tested on HeNC2 and GG2EE cells as follows: Both cell lines were maintained in RPMI-1640 with 10% FCS. At time of subculture, cells were plated at 1×105 cells/well in a flat-bottomed 96-well plate. After 1 hour of incubation, the media was replaced, and stimulants were added at several concentrations. The cells were incubated for about 16-18 hours, after which the supernatants were removed for subsequent assay. HeNC2 and GG2EE supernatants were assayed for TNF release by ELISA as described earlier, and NO by Griess assay. The results demonstrate that emulsan induces TNF release from these cells in an LPS-independent manner. There was no nitrite detected in these samples (FIG. 6).

The abbreviations used in FIGS. 7A and 7B are as follows. SL is crude sophorolipid which is a glycolipid in the disaccharide sophorose is linked glycosidically to the hydroxyl group at the penultimate carbon of C16 to C19 chain length fatty acids; C2 is ethylester of sophorolipid; and Ac is diacetylated ethyl ester of the sophorolipid—these were isolated from the yeast, Candida bombicola and enzymatically or chemically modified] did not elicit TNF release from the macrophages under the same experimental conditions. Thus, the response is specific to emulsan and not microbial glycolids in general.

Furthermore, since the responses of both the LPS non-responsive and responsive macrophages were the same, the emulsan was not contaminated with significant levels of bacterial LPS. Otherwise a dose-dependent effect on the HeNC2 cells (responsive) would have been expected that would not have been seen with the non-responsive cell line (GG2EE) as seen with bacterial LPS in FIG. 2B. In addition, these results suggest that emulsan is inducing TNF release from macrophages through a different mechanism that LPS since the GG2EE cells, which are deficient in the LPS-response pathway, released roughly the same amount of TNF as the HeNC2 cells.

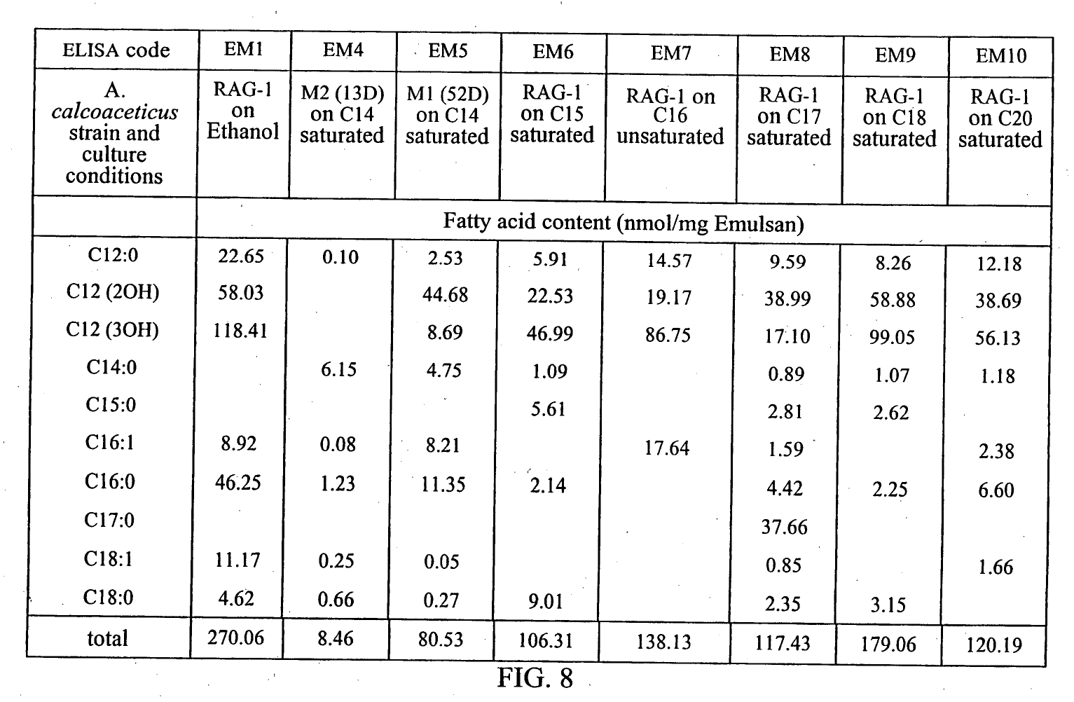

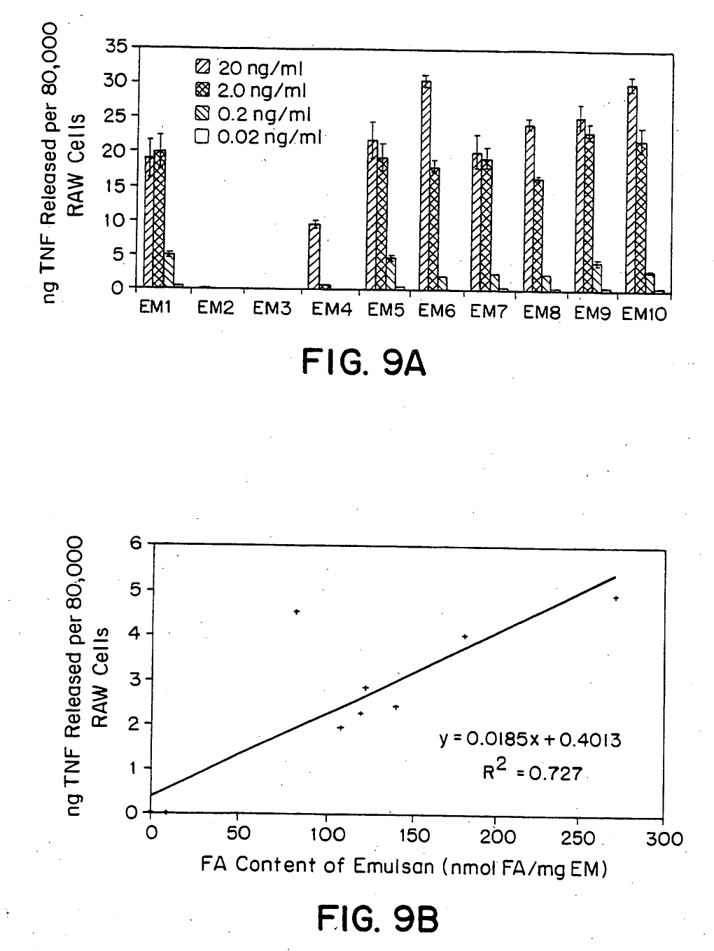

In a second set of studies, a series of emulsan analogs, shown in FIG. 8, were screened in the same macrophage assay as described above. The primary variant with these analogs was the degree of substitution of fatty acids (FIGS. 9A and 9B). There was clear correlation between lower TNF release with lower degree of substitution, and this response disappears completely when all fatty acids are removed from the polysaccharide backbone (EM2 and EM3 in FIG. 9A).

Adjuvant Activity of Emulsan—Adjuvanticity was assayed in vivo with mice with the following conditions and controls:

TABLE 5
Experimental Groups Used in In Vivo Immunizations

| Group | Adjuvant | Antigen |
|---|---|---|
| 1 | None | 100 μg KLH-DNP |
| 2 | 200 μg Crude Emulsan | None |
| 3 | 200 μg Deproteinized Emulsan | None |
| 4 | 20 μg Crude Emulsan | 100 μg KLH-DNP |
| 5 | 200 μg Crude Emulsan | 100 μg KLH-DNP |
| 6 | 20 μg Deproteinized Emulsan | 100 μg KLH-DNP |
| 7 | 200 μg Deproteinized Emulsan | 100 μg KLH-DNP |
| 8 | 100 μl Complete Freund's Adjuvant/ | 100 μg KLH-DNP |
| | Incomplete Freund's Adjuvant* | |

*CFA was used as adjuvant in primary immunization, and IFA was used at boost.

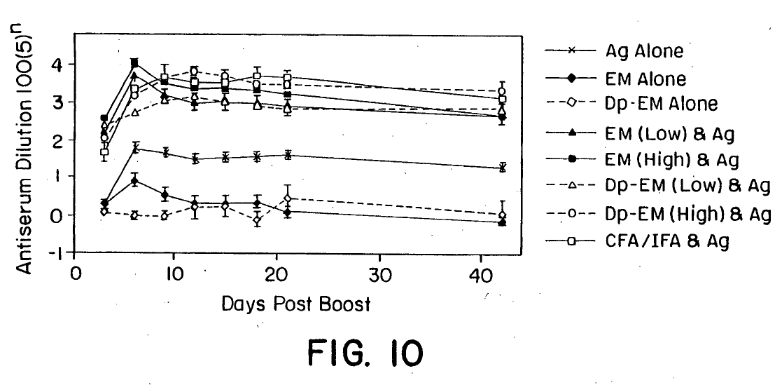

Data points for each mouse were fit to a sigmoidal curve (Graphpad Prizm) and the dilution at which absorbance was twice baseline was determined. The results indicate that both preparations of emulsan (with and without associated protein) have significant adjuvant activity which is on the same order as Freund's adjuvants (FIG. 10). The relative contribution of T-helper sub-types in the immune response in these immunized animals was also examined.

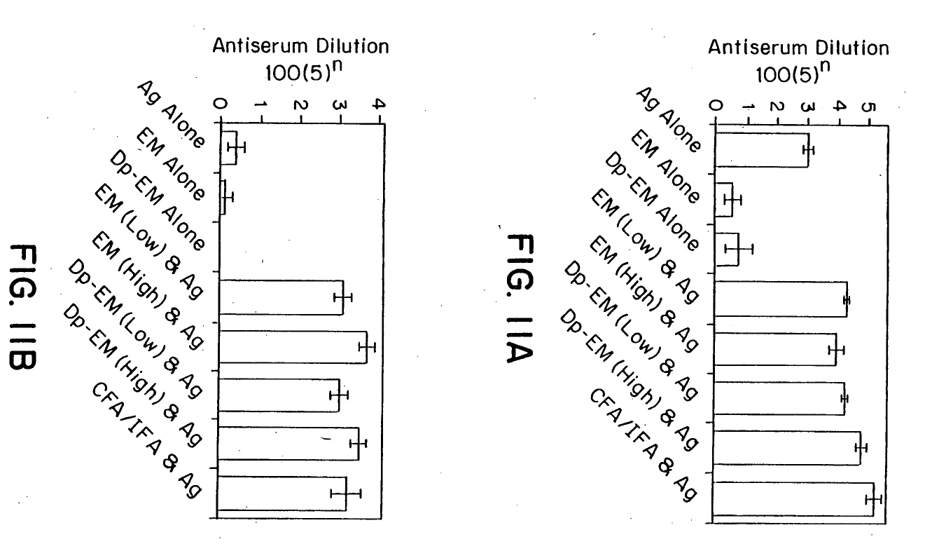

FIGS. 11A and 11B illustrates the relative proportions of the IgG isotypes IgG2a and IgG1 as a measure of the contribution of T-helper type-1 and T-helper type-2 specific isotypes, respectively. The ELISA was conducted as above with the substitution of HRP-conjugated isotype-specific antibodies (Accurate) for the goat anti-mouse antibody. The dilution at which the absorbance was twice baseline was determined as for the total antibody titers. In order to compare the relative contribution of a T helper type 1 versus a T helper type 2 response, the ratio of the IgG2a dilution to the IgG1 dilution was determined. This analysis allowed comparison of relative levels of each isotype between groups. Thus, the adjuvants induced higher IgG1 and IgG2a levels than antigen alone.

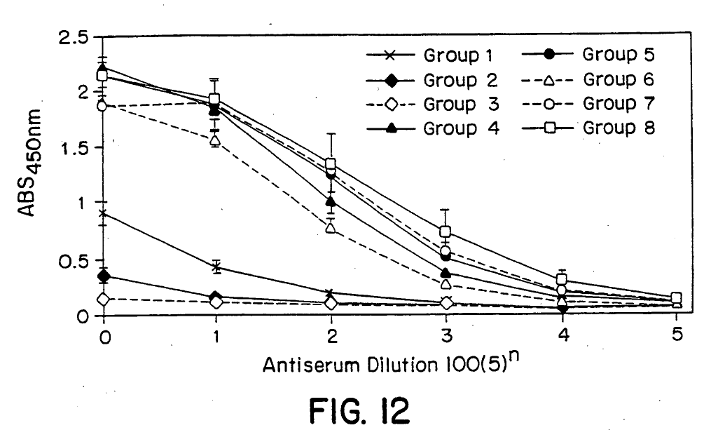

Immunization of mice with preparations of emulsan in various treatment groups as described in Table 5 resulted in the presence of antibodies in the serum of mice (FIGS. 10 and 12). Thus, emulsan are effective in electing an immune response in a mammal which is not accompanied by cytotoxicity.

In Vivo Toxicity of Emulsan

All of the mice immunized using emulsan as an adjuvant in these experiments were alive longer than 5 months after the initial immunization. This is significant considering that twenty of these mice were injected with 200 μg of emulsan (10 with crude, 10 with deproteinized), especially when this is compared to toxicity data for some saponin-based adjuvants where certain fractions are lethal within 3 days at similar concentrations (Kensil, C. R., et al., In Vaccines 92, F. Brown, et al., eds. (Cold Spring Harbor Press) pp.35-40 (1992), the teachings of which are incorporated herein by reference in their entirety). Mice injected with emulsan showed no more discomfort than the mice injected with antigen alone, while the mice injected with Freund's adjuvants were obviously in severe discomfort immediately following immunization.

Exposure to 200 μg of emulsan did not increase the frequency of chronic disease over controls. Mice were euthanized and examined at approximately 42 weeks of age (36 weeks following primary immunization). Tissue samples were taken from the lungs, liver, pancreas, mesenteric lymph node, heart, and kidney. All mice, including those receiving antigen alone (Group 1) displayed mild-to-moderate lymphatic hyperplasia in the mesenteric lymph nodes. One of the six emulsan-treated animals showed evidence of a widespread lymphoma. However, one of the two control animals (no emulsan) developed a well-differentiated papillary adenocarcinoma. These conditions can indicate age than treatment as the average life span of BALB\c female mice is approximately 80 weeks (Storer, J. B., J. Gerontol. 21:404-409 (1966), the teachings of which are incorporated herein by reference in their entirety). Thus, the preliminary toxicity data demonstrate that the mice immunized with emulsan presented no acute toxicity, nor increased histopathology compared to control animals.

Equivalents

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. An immunization formulation, comprising:
a) an antigen; and
b) an emulsan adjuvant.

2. The immunization formulation of claim 1, wherein the emulsan adjuvant is secreted from Acinetobacter calcoaceticus.

3. The immunization formulation of claim 2, wherein the emulsan adjuvant is secreted from Acinetobacter calcoaceticus RAG-1.

4. The immunization formulation of claim 1, wherein the emulsan adjuvant is secreted by a mutant of Acinetobacter calcoaceticus.

5. The immunization formulation of claim 4, wherein the emulsan adjuvant is secreted by a transposon mutant of Acinetobacter calcoaceticus.

6. The immunization formulation of claim 1, wherein the emulsan adjuvant has an average fatty acid chain length in a range of between about 10 carbons and about 20 carbons.

7. The immunization formulation of claim 1, wherein the emulsan adjuvant has a fatty acid density in a range of between about 25 nmol/mg emulsan and about 900 nmol/mg emulsan.

8. The immunization formulation of claim 1, wherein the emulsan adjuvant has an amount of saturated bonds in fatty acids of the analog in a range of between about 80 mole % and about 100 mole %.

9. The immunization formulation of claim 1, wherein the emulsan adjuvant has an amount of hydroxylated fatty acids in a range of up to 65 mole %.

10. The immunization formulation of claim 1, wherein the emulsan adjuvant is formed by feeding Acinetobacter calcoaceticus or a mutant thereof a compound selected from the group consisting of fatty acids, fatty acid salts, hydroxylated fatty acid salts and complex carbon sources that include fatty acids, said group having a carbon chain length in a range of between about 10 carbons and about 20 carbons.

11. The immunization formulation of claim 1, wherein the antigen is selected from the group consisting of peptides, polypeptides, viruses, bacteria, fungi, and parasites.

12. The immunization formulation of claim 11, wherein the antigen is dinitrophenol coupled to keyhole limpet hemocyanin.

13. A formulation comprising an antigen and an emulsan adjuvant for stimulating an immune response in an organism.
